(19) United States
(12) Patent Application Publication
(10) Pub. No.: US 2012/0282202 A1
(43) Pub. Date: Nov. 8, 2012

(54) AQUEOUS COMPOSITION FOR TOPICAL APPLICATION, METHOD OF PREPARATION, USES AND DEVICE

(76) Inventor: Miriam Clapposen, Amsterdam (NL)

(21) Appl. No.: 13/504,343

(22) PCT Filed: Oct. 27, 2010
(86) PCT No.: PCT/NL2010/050720

§ 371 Date: Jul. 24, 2012

(30) Foreign Application Priority Data
Oct. 27, 2009 (NL) 2003711

(51) Int. Cl. A61K 31/22; A61P 31/10

(52) U.S. Cl. 424/61; 514/546

(57) ABSTRACT

An aqueous composition for topical application includes at least 60% w/w of a C1-C4 alkyl lactate ester, at least 1% w/w water, having a pH in the range from 1-6, and an effective amount of at least one physically acceptable antioxidant. A method is provided for the preparation, and the use of the composition for the treatment of fungus infections of nails and/or skin, in particular onychomycosis and tinea pedis, and an applicator device for performing such treatment.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The invention relates to an aqueous composition for topical application, a method for the preparation, and the use of the composition for the treatment of fungus infections of nails and/or skin, in particular onychomycosis and tinea pedis. The invention also provides an applicator device.

2. Description of Related Art Including Information Disclosed Under 37 CFR 1.97 and 37 CFR 1.98

According to the International patent application WO2008128627 in the name of BioEqual, alkyl esters of lactic acid, malic acid, tartaric acid and citric acid are known in formulations for treating nail fungus. However, the formulations described therein will have to be water-free, as these formulations are said to be unstable once water is added.

### BRIEF SUMMARY OF THE INVENTION

It is an object of the invention to provide an aqueous composition for topical application, which is relatively stable.

The invention provides an aqueous composition for topical application, comprising at least 60% w/w of a C1-C4 alkyl lactate ester, at least 1% w/w water, having a pH in the range from 1-6, and an effective amount of at least one physically acceptable antioxidant. Such a composition is useful as a topical application, in particular for the treatment of fungus infections of nails and/or skin, in a medicament against onychomycosis and/or in a medicament against tinea pedis. The composition is relatively stable due to the addition of the antioxidant, combined with the pH in the range of 1-6. Another advantage is that the formulation according to the invention allows for a relatively low amount of C1-C4 alkyl lactate esters. These esters are relatively expensive, and also from an environmental point of view it is advantageous to lower the amount of C1-C4 alkyl lactate ester. A further advantage is that a lower amount of C1-C4 alkyl lactate esters reduces the chance of irritation when applied to a person having a sensitive skin. The composition is preferably a homogeneous solution.

The C1-C4 alkyl lactate ester includes methyl, ethyl, n-propyl, isopropyl, n-butyl, isobutyl and tert-butyl esters. The at least 1% w/w water may add up to a 100% of the total composition, but the composition may also include other additional ingredients. Preferably, the amount of water is at least 10%, for instance 20% or 30%.The pH is determined by well known methods, such as pH-sensitive indicator paper, or electronic pH meters using a glass electrode. Various antioxidants may be used, preferably antioxidants having a sufficient solubility in the aqueous alkyl lactate ester/water composition. The efficient amount of antioxidant depends on the type of oxidant, and should be sufficient to achieve a good stability. Good stability is defined as a composition wherein, after storage during 6 months at 40° C. , at least 90% of the C1-C4 alkyl lactate ester is still present compared to the same composition lacking the antioxidant. The stability of the composition can be monitored by pH measurements, as well as chromatographic methods, for instance gas chromatography (GC) or liquid phase chromatography methods such as HPLC.

In a preferred embodiment, the alkyl lactate ester is lactic acid ethyl ester. Lactic acid ethyl ester was shown to be particularly suitable for applications against nail and skin treatments. Compared to other C1-C4 alkyl lactate esters, the ethyl ester showed a relatively good stability in aqueous compositions according to the invention having a pH in the range 1-6, also comprising a suitable antioxidant.

Preferably, the composition also comprises lactic acid in a molecular ratio of at least 1:100 with respect to the C1-C4 alkyl lactate ester, preferably at least 1:20, most preferably at least 1:10. Such a composition showed improved stability of the C1-C4 alkyl lactic acid ester in the pH-range 1-6. Lactic acid refers to the equivalent amount of the free acid present in the composition. Depending on pH, part of the lactic acid is present in the lactate form.

It is preferred if the pH is in the range from 2.5 to 3.5. At this pH range, stability of the composition was shown to be better than lower or higher pH compositions. In particular, the ethyl lactic acid ester benefitted from increased stability in this pH range. As an additional advantage, the compositions having such a low pH in the range of 2.5-3.5 work particularly well against fungal infections. In order to further improve the antifungal effect, optionally a pharmacologically active anti-fungal agent may be added to the preparation.

It is preferred if the pH is stabilised by a pH buffer. A buffered pH shows a more reliable stability of the composition, leading to an increased effective shelf life.

In preferred embodiment, the pH buffer is based on a water-soluble carboxylic acid. Carboxylic acids show a good compatibility with C1-C4 alkyl lactate esters, and are easily miscible with such esters. Suitable carboxylic acids include lactic acid, malic acid, tartaric acid and citric acid.

Preferably, the pH buffer is a lactate buffer. Such a buffer, based on lactic acid, shows an excellent compatibility with the C1-C4 alkyl lactate ester compositions.

It is advantageous if the antioxidant comprises at least one antioxidant selected from the group consisting of ascorbic acid (vitamin C), sodiumbisulfite, tocopherol (vitamin E), L-Ergothioneine, Resveratrol, Alpha Lipoic Acid. Preferably, the antioxidant is dissolved in the composition. These antioxidants showed excellent improvement in stability of the compositions compared to the same composition without the antioxidant. One or more of these antioxidants may used as a combination.

Most preferably, the antioxidant comprises tocopherol (vitamin E). Tocopherol was shown to function particularly well as an antioxidant in C1-C4 alkyl lactate ester compositions, generally showed a lack of side-effects and is considered to be a very safe antioxidant.

The composition preferably comprises tocopherol in a concentration of at least 0.1% w/w, most preferably between 0.5-2%. In this range, tocopherol dissolves well in the C1-C4 alkyl lactate ester and shows an excellent improvement in stability.

The invention also provides a method for the preparation of a composition according to the invention, comprising the steps of mixing at least 60% w/w of a C1-C4 alkyl lactate ester, at least 1% w/w water, and an effective amount of at least one pharmaceutically acceptable antioxidant, and adjusting the pH of the water to the range from 1-6, preferably from 2.5-3.5. The pH maybe adjusted by titration, for instance using sodium hydroxide and/or hydrochloric acid solutions, or by the preparation of a buffer. The pH should be checked after mixing.

The invention also provides the use of a composition according to the invention for the preparation of a product for the treatment of fungus infections of nails and/or skin. It is particularly advantageous if the composition also includes at least 1% of essential oils derived from plants. In particular oils derived from tea tree oil, lavendula and callitris intratropica showed advantageous effects. Such oils may be used as a mixture.

In particular, the invention provides the use of a composition according to the invention for the preparation of a medicament against onychomycosis. Onychomycosis includes dermatophytes; Trichophyton rubrum is the most common dermatophyte involved in onychomycosis. Other dermatophytes that may be treated using the composition according to the invention are Trichophyton interdigitale, Epidermophyton floccosum, Trichophyton violaceum, Microsporum gypseum, Trichophyton tonsurans, Trichophyton soudanense and the cattle ringworm fungus Trichophyton verrucosum.

Also, the composition according to the invention may be used for the preparation of a medicament against tinea pedis.

The invention further provides a device, comprising a container comprising a composition according to the invention, and an applicator connected to the container, wherein the applicator is adapted to apply the composition from the container to a human nail or human skin.

### BRIEF DESCRIPTION OF THE DRAWINGS

FIG. 1 shows an applicator for applying a composition according to the invention.

### DETAILED DESCRIPTION OF THE INVENTION

The invention will now be further elucidated by the following non-limiting examples.

Applicator Device:

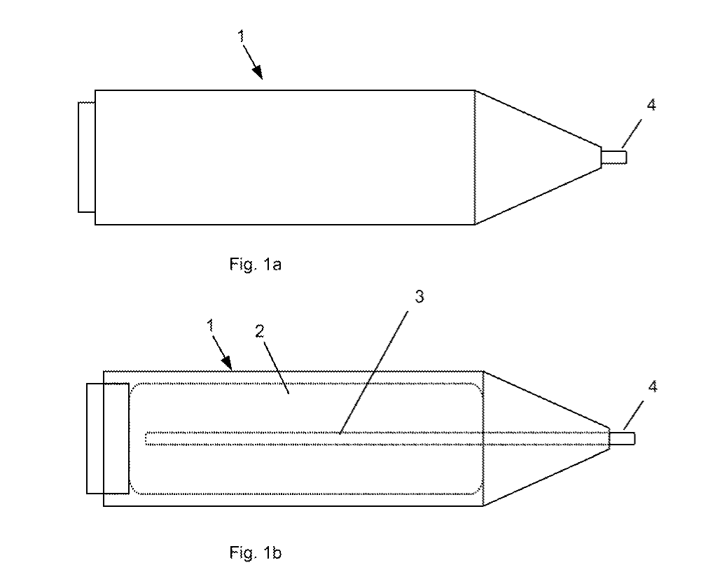

FIG. 1 shows a pen application device 1, somewhat resembling a felt-tipped type marker. FIG. 1a shows the outer appearance of the applicator 1, whereas FIG. 1b shows the cross section. The device 1 comprises a container 2 containing a liquid composition according to the invention. The device is pencil-shaped, and suitable to be hand-held. The container is provided with an absorbing element 3 made of a liquid-absorbing material capable of capillary action. This absorbing element dips into the liquid composition and extends from the inner part container to the outside of the container. The liquid is contained in the container and can be applied via an applicator tip 4 connecting to or made out of the distal end of the absorbing element extending out of the container. Due to capillary action, the tip 4 remains moist with the liquid product. For such an applicator device, the viscosity of the liquid composition will have to be sufficiently low. The device may be provided with a cap to prevent volatile solvents of the composition to evaporate and dry the tip out. The moist tip 4 is contacted with the skin part to be treated, preferably while applying some pressure, in order to apply the composition from the container.

The applicator may be filled with a liquid composition according to any of the following non-limiting examples.

### EXAMPLE 1

A composition was prepared by well know mixing techniques (percentages by weight). The pH was adjusted by titration with aqueous 1M hydrochloric acid and/or 1M sodium hydroxide under stirring.

Lactic acid 7%

lactic acid ethyl ester 85%

tocopherol 1%

Water 7%

pH: 3.5

This composition is suitable for the treatment of fungus infections of nails and/or skin, in particular various forms of onychomycosis as well as tinea pedis.

### EXAMPLE 2

A composition was prepared by well know mixing techniques (percentages by weight). First a lactate buffer was prepared by dissolving lactic acid in water and adjusting the pH titration with aqueous 1M hydrochloric acid and/or 1M sodium hydroxide under stirring to reach the preferred value. The lactate buffer was mixed with lactic acid ethyl ester and an antioxidant.

Lactic acid/sodium Lactate (pH buffer)* 20%

lactic acid ethyl ester 65%

tocopherol 0.7%

Water 14.3%

*percentage is referring to the amount of lactic acid present, either as lactic acid or lactate. pH: 2.0

This composition is suitable for the treatment of fungus infections of nails and/or skin, in particular various forms of onychomycosis as well as tinea pedis.

### EXAMPLE 3

A composition was prepared by well know mixing techniques (percentages by weight). The pH was adjusted by titration with aqueous 1M hydrochloric acid and/or 1M sodium hydroxide under stirring.

Tea Tree Oil 1%

Lavendula 2%

Callitris Intratropica Oil 2%

Lactic acid 2%

lactic acid ethyl ester 80%

sodium bisulfite 0.2%

Water 12.8%

pH: 4.7

This composition is suitable for the treatment of fungus infections of nails and/or skin, in particular various forms of onychomycosis as well as tinea pedis.

### EXAMPLE 4

A composition was prepared by well know mixing techniques (percentages by weight). The pH was adjusted by titration with aqueous 1M hydrochloric acid and/or 1M sodium hydroxide under stirring.

Amorolfine 5%

Lactic acid 2%

lactic acid ethyl ester 80%

sodium bisulfite 0.2%

Water 12.8%

pH: 4.7

This composition is suitable for the treatment of fungus infections of nails and/or skin, in particular various forms of onychomycosis. Amorolfine is a known antifungal agent, which works particularly well in a lactic acid ethyl ester composition according to the invention.

1. Aqueous composition for topical application, comprising
at least 60% w/w of a C1-C4 alkyl lactate ester,
at least 1% w/w water, having a pH in the range from 1-6,
and an effective amount of at least one physically acceptable antioxidant.

2. Composition according to claim 1, wherein the alkyl lactate ester is lactic acid ethyl ester.

3. Composition according to claim 1, wherein the composition also comprises lactic acid in a molecular ratio of at least 1:100 with respect to the C1-C4 alkyl lactate ester, preferably at least 1:20, most preferably at least 1:10.

4. Composition according to claim 1, wherein the pH is in the range from 2.5 to 3.5.

5. Composition according to claim 1, wherein the pH is stabilised by a pH buffer.

6. Composition according to claim 5, wherein the pH buffer is based on a water-soluble carboxylic acid.

7. Composition according to claim 5, wherein the pH buffer is a lactate buffer.

8. Composition according to claim 1, wherein the antioxidant comprises at least one antioxidant selected from the group consisting of ascorbic acid (vitamin C), sodiumbisulfite, tocopherol (vitamin E), L-Ergothioneine, Resveratrol, Alpha Lipoic Acid.

9. Composition according to claim 8, wherein the antioxidant comprises tocopherol (vitamin E).

10. Composition according to claim 8, wherein the composition comprises tocopherol in a concentration of at least 0.1% w/w, preferably between 0.5-2%.

11. Method for the preparation of a composition according to claim 1, comprising the steps of mixing at least 60% w/w of a C1-C4 alkyl lactate ester, at least 1% w/w water, and an effective amount of at least one pharmaceutically acceptable antioxidant, and adjusting the pH of the water to the range from 1-6, preferably from 2.5-3.5.

12. Use of a composition according to claim 1, for the preparation of a product for the treatment of fungus infections of nails and/or skin.

13. Use of a composition according to claim 1, for the preparation of a medicament against onychomycosis.

14. Use of a composition according to claim 1, for the preparation of a medicament against tinea pedis.

15. Device, comprising a container comprising a composition according to claim 1, and an applicator connected to the container, wherein the applicator is adapted to apply the composition from the container to a human nail or human skin.
